(19) United States
(12) Patent Application Publication
(10) Pub. No.: US 2025/0297275 A1
(43) Pub. Date: Sep. 25, 2025

(54) TOMATO PLANT RESISTANT TO TOMATO SPOTTED WILT VIRUS

(71) Applicant: Enza Zaden Beheer B.V., Enkhuizen (NL)

(72) Inventors: Marieke Ykema, Enkhuizen (NL); Hille-Jan Van Zwol, Enkhuizen (NL); Sergio De La Fuente Van Bentem, Enkhuizen (NL); Geert Johannes De Boer, Enkhuizen (NL)

(21) Appl. No.: 18/843,548

(22) PCT Filed: Mar. 4, 2022
(86) PCT No.: PCT/EP22/55586

§ 371 Date: Sep. 3, 2024

(51) Int. Cl. C12N 15/82; A01H 1/00; A01H 5/08; A01H 6/82; C07K 14/415

(52) U.S. Cl. CPC C12N 15/8283; A01H 1/126; A01H 5/08; A01H 6/825; C07K 14/415

(57) ABSTRACT

The present invention relates to a plant of the S. lycopersicum species that is resistant to Tospovirus, wherein the plant comprises a TSWV resistance gene. More specifically the invention relates to tomato plants (S. lycopersicum) that are resistant to Tomato Spotted Wilt Virus (TSWV). The present invention further relates to a resistance gene or genomic sequence providing resistance to Tospovirus. Furthermore, the present invention relates to methods for providing a S. lycopersicum plant that is resistant to Tospovirus.

The present invention relates to a plant of the S. lycopersicum species that is resistant to Tospovirus, wherein the plant comprises a TSWV resistance gene. More specifically the invention relates to tomato plants (S. lycopersicum) that are resistant to Tomato Spotted Wilt Virus (TSWV). The present invention further relates to a resistance gene or genomic sequence providing resistance to Tospovirus. Furthermore, the present invention relates to methods for providing a S. lycopersicum plant that is resistant to Tospovirus.

Tospoviruses are severe plant pathogens with a broad host range, infecting more than 1000 plant species belonging to 80 different families. These include both food crops including watermelons, capsicums, zucchinis and tomatoes. Transmission of Tospoviruses usually occurs via thrips and Tospoviruses are prevalent in warm climates in regions with a high population of thrips. The Tospovirus genus includes Tomato spotted wilt virus (TSWV), Groundnut ringspot virus (GRSV), Groundnut bud necrosis virus (GBNV), Capsicum chlorosis virus (CaCV), and Tomato chlorotic spot virus (TCSV). TSWV is described as an emerging viral disease of plants and causes serious losses in economically important crops and it is one of the most economically devastating plant viruses in the world. Infection with Tospoviruses results in spotting and wilting of the plant, reduced vegetative output, and eventually death. The most efficient method of containing this disease is genetic resistance. There are no antiviral cures for plants infected with a Tospovirus, and infected plants should be removed from a field and destroyed in order to prevent the spread of the disease. Tospoviruses cause an estimated annual crop loss of one billion U.S. dollars and are considered one of the most devastating plant viruses worldwide.

Plants use both cell surface-resident pattern recognition receptors (PRRs) and intracellular nucleotide binding leucine-rich repeat (NLR) receptors to detect various pathogens, including Tospoviruses. PRRs recognize conserved pathogen-associated molecular patterns (PAMPs) to provide broad-spectrum resistance, whereas NLRs detect pathogen strain-specific effectors and confer race specific resistance. A plant NLR recognizes a small, conserved peptide of phylogenetically related pathogens. The Tomato Sw-5 resistance gene is an NLR (Brommonschenkel and Tanksley, 1997) and is an effective dominant resistance gene that provided broad-spectrum resistance to tomato-infecting tospoviruses including Tomato spotted wilt virus (TSWV), Groundnut ringspot virus (GRSV), and Tomato chlorotic spot virus (TCSV). The Sw-5 protein recognizes the TSWV movement protein, which is required for cell-to-cell movement and systemic infection of tospoviruses.

In the battle against Tospovirus, resistance was introduced in tomatoes by introgression of the Sw-5 gene, resulting in resistance. However, recently new strains of Tospovirus have emerged as resistance is overcome and Sw-5 resistance-breaking Tospovirus species have been reported in Australia, US, Europe and Mexico, more specifically TSWV strains. Although Sw-5 resistance is still very useful, those resistance breaking strains of TSWV have become more widespread worldwide and result in a growing problem. Therefore, new resistance genes need to be identified and/or combined to provide resistant crops, especially against the new TSWV strains.

Considering the above, there is a need in the art for TSWV resistant tomato plants, more specifically TSWV resistant S. lycopersicum. More specifically, there is a need for TSWV resistant plants that are resistant to the resistance-breaking isolates of TSWV. In addition, there is a need in the art to provide methods and means for providing TSWV resistant S. lycopersicum plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a plant of the S. lycopersicum species that is resistant to a Tospovirus, wherein the plant comprises a TSWV resistance gene that encodes for a TSWV resistance protein, wherein the protein has at least 90%, at least 91%, at least 92%, at least 93%, at least 94%, preferably at least 95%, more preferably at least 98%, even more preferably at least 99%, most preferably 100% amino acid sequence identity with SEQ ID No.1. The plant may comprise a TSWV resistance gene that encodes for a TSWV resistance protein of Solanum (S.) peruvianum. It is predicted that the TSWV resistance gene encodes for an NBS-LRR resistance protein. Pathogen recognition by plants takes place via two relevant groups of host receptors involving two main types of proteins, namely Receptor-like kinases or proteins (RLK or RLP) and nucleotide-binding site leucine-rich repeat proteins (NBS-LRR resistance proteins). The first group are pattern recognition receptors (PRR) specializing in the recognition of pathogen associated molecular patterns (PAMPS). RLPs or RLKs are attached to the cell membrane and are extracellular immune receptors. Plant RLKs are involved in plant-pathogen interaction and defence responses and plant receptor kinases (PRKs) can be defined as proteins that contain an extracellular domain, a single-pass transmembrane domain and a cytoplasmic serine/threonine (ser/thr) protein kinase domain. Plant LRR-RLKs (leucine rich-repeat receptor-like kinase) possess a functional cytoplasmic kinase domain, and all of the plant LRR-RLKs analysed to date possess ser/thr kinase activity. The resistance to pathogens provided by these receptors is called PAMP-triggered immunity (PTI). The other group mainly comprises intracellular receptors called resistance proteins (R proteins). The majority of disease resistance genes in plants encode nucleotide-binding site leucine-rich repeat proteins, also known as NBS-LRR proteins. These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. The majority of the identified genomic sequences that provide Tospovirus resistance comprise multiple LRR domains. It is thought that these domains determine effector recognition and therefore disease susceptibility/resistance.

Pathogens develop counter strategies to overcome PTI through modifying or changing PAMPs or MAMPs. Alternatively, plants have developed another way to recognize pathogens by triggering a faster and stronger secondary defence response known as effector-triggered immunity (ETI). ETI is mediated by R proteins which recognize so called pathogen effectors and which results in localized cell death around the site of infection. The availability of a newly identified resistance gene and/or genomic regions encoding NBS-LRR proteins and/or plant receptor kinases will decrease the chances of the pathogen overcoming the resistance, or when combined with other resistance genes, disease resistance may even be further improved.

According to a preferred embodiment, the present invention relates to the plant, wherein the TSWV resistance gene comprises a coding sequence that has at least 90%, at least 91%, at least 92%, at least 93%, at least 94%, preferably at least 95%, more preferably at least 98%, even more preferably at least 99%, most preferably 100% sequence identity with SEQ ID No. 2 and/or SEQ ID No. 4. SEQ ID No. 2 is the coding sequence of the TSWV resistance gene, and SEQ ID No. 4 is similar to SEQ ID No. 2, but in addition also comprises the UTR sequence of the TSWV resistance gene.

According to another preferred embodiment, the present invention relates to the plant, wherein the plant comprises a genomic sequence having at least 90%, preferably at least 95% sequence identity with SEQ ID No. 3. The genomic sequence comprises multiple sequences that have homology with sequences that encode for NBS-LRR resistance proteins. The genomic sequences encode for one or more genes and/or genetic elements that provide resistance to Tospovirus. Sequences have been examined on gene homology using the public database of the National Center for Biotechnology Information (NCBI). Three genomic sequences have homology with sequences that encode for NBS-LRR resistance proteins. One of these specific sequences proved, after VIGS silencing experiments, to comprise the TSWV resistance gene.

According to a preferred embodiment of the present invention the present plants detailed above are not plants exclusively obtained by means of an essentially biological process. Although the present genomic regions or fragments can be introduced into tomato plants by introgression, because the nucleotide sequences of the present genomic fragments are known, these genomic fragments, for example, can be artificially constructed in yeast and subsequently allowed to recombine with susceptible tomato genomes. Alternatively, these genomic regions or fragments can be amplified by long-range PCR amplifications and the resulting amplification fragments can be transformed into tomato cells in a single step or in a series of transformations ultimately resulting in the present tomato plants. The present genomic fragments, completely or in parts later to be reassembled, can also be isolated from gels or columns for example after restriction digestion, and subsequently transformed into tomato cells. Furthermore, mutations, deletions or insertions in the genome can be obtained via EMS mutagenesis, and/or CRISPR technology. Yet alternatively, the genomic fragments of interest can be introduced into a vector under a (strong) promotor. Subsequently, susceptible plants can be transformed with the vector and the sequence of interest would be expressed resulting in resistance. These techniques are readily available for the skilled person. Construction of artificial chromosomes comprising the present genomic fragments is also contemplated within the context of the present invention.

According to another preferred embodiment, the present invention relates to the plant, wherein the Tospovirus is one or more selected from the group consisting of Tomato spotted wilt virus (TSWV), Groundnut ringspot virus (GRSV), Groundnut bud necrosis virus (GBNV), Capsicum chlorosis virus (CaCV), and Tomato chlorotic spot virus (TCSV), preferably TSWV.

According to yet another preferred embodiment, the present invention relates to the plant, wherein the plant is resistant to Tomato Spotted Wilt Virus (TSWV).

According to yet another preferred embodiment, the present invention relates to the plant, wherein the TSWV comprises the C118Y mutation and/or T120N mutation in the non-structural movement (NSm) protein of TSWV. It is known that the present TSWV resistance breaking strains, for example the virus isolate AJ137, comprise the C118Y mutation and/or T120N mutation. The plant of present inventions shows to be resistant to this specific TSWV resistance breaking virus comprising the C118Y mutation. Two mutations have been described, both leading to amino acid changes in the non-structural movement (NSm) protein of TSWV (GenBank: ATL64765.1): C118Y and the T120N. These amino acids are located in the 21-amino acid region of the NSm protein that is recognized by the Sw-5 protein (Zhu et al., 2017). Increased cultivation of Sw-5 varieties has recently led to the emergence of resistance breaking strains of TSWV in California, and all contained the C118Y mutation (Batuman et al., 2018).

According to yet another preferred embodiment, the present invention relates to the plant, wherein the genomic sequences and/or TSWV resistance gene is homozygous present in the genome of said plant. From the experimental data it can be concluded that the resistance is incomplete dominant and that the TSWV resistance gene and/or genomic sequence must be homozygous present in the genome of the plant to provide full resistance against the Tospovirus.

According to another preferred embodiment, the present invention relates to the plant, wherein said TSWV resistance gene and/or genomic sequences are as found in the deposit accession number NCIMB 43771. Solanum lycopersicum seeds were deposited on 7 May 2021 at NCIMB Ltd, Ferguson Building, Craibstone Estate Bucksburn, AB21 9YA Aberdeen, United Kingdom. These seeds can be obtained by crossing with a wild tomato plant of Solanum peruvianum. obtained from the Tomato Genetics Resource Center (TGRC), Department of Plant Sciences, University of California, Davis, USA.

According to a preferred embodiment, the present invention relates to the plant, wherein the plant further comprises an Sw-5 resistance gene that encodes for Sw-5 protein having at least 95% amino acid sequence identity, more preferably at least 98%, even more preferably at least 99%, most preferably 100% sequence identity with SEQ ID No.11. The coding sequence encoding the Sw-5 protein is included herein as SEQ ID No. 12.

The present invention, according to a second aspect, relates to plants, plant parts, tissues, cells, and/or seeds, including offspring plants derived from the plant of present invention.

The present invention, according to a further aspect, relates to a resistance gene (TSWV resistance gene) for providing resistance to a Tospovirus in a S. lycopersicum plant, wherein said resistance gene is represented by a coding sequence having at least 90% nucleotide sequence identity with SEQ ID No.2 and/or SEQ ID No.4. The resistance gene may be part of a composition where the gene or genomic sequence is combined with other sequences e.g., in a vector or gene expression construct.

The present invention, according to a further aspect, relates to a genomic sequence for providing resistance to a Tospovirus in a S. lycopersicum plant, wherein the genomic sequence has at least 90% sequence identity, at least 91%, at least 92%, at least 93%, at least 94%, preferably at least 95%, more preferably at least 98%, even more preferably at least 99%, most preferably 100% sequence identity with SEQ ID No. 3.

The present invention, according to a further aspect, relates to a resistance gene or genomic sequence for providing resistance to a Tospovirus in a S. lycopersicum plant, wherein the Tospovirus is one or more selected from the group consisting of Tomato spotted wilt virus (TSWV), Groundnut ringspot virus (GRSV), Groundnut bud necrosis virus (GBNV), Capsicum chlorosis virus (CaCV), and Tomato chlorotic spot virus (TCSV), preferably TSWV. According to a preferred embodiment of present invention, the resistance gene, genomic sequence or resistance locus provides resistance to a TSWV. The TSWV preferably comprises the C118Y mutation and/or T120N mutation in the non-structural movement (NSm) protein of TSWV.

The present invention, according to a further aspect, relates to a method for providing a plant of the S. lycopersicum species that is resistant to Tospovirus, wherein the method comprises the steps of;
a) selecting a tomato plant that is resistant to Tospovirus, wherein said selection comprises establishing the presence of the TSWV resistance gene or TSWV genomic sequence of present invention,
b) transferring the identified genomic sequence or locus of step a) into a S. lycopersicum plant thereby conferring Tospovirus resistance to said S. lycopersicum plant.
   Transferring can for example be done by crossing a selected resistant tomato plant (for example S. peruvianum) with a S. lycopersicum. Subsequently, a Tospovirus resistant S. lycopersicum plant can be selected.

According to yet another preferred embodiment, the present invention relates to the method, wherein said tomato plant that is resistant to Tospovirus is an S. lycopersicum plant or S. peruvianum plant, preferably S. peruvianum.

According to another preferred embodiment, the present invention relates to the method, wherein after step b) a first S. lycopersicum plant is selected that is resistant to Tospovirus and is crossed with a second S. lycopersicum plant that is not resistant to Tospovirus, and subsequently selecting S. lycopersicum plants that are resistant to Tospovirus.

According to a preferred embodiment, the present invention relates to the method, wherein in step a) establishing the presence of the resistance gene (TSWV resistance gene) or resistance conferring genomic sequence in a tomato plant is performed by one or more markers selected from the group consisting of SEQ ID No: 5, SEQ ID No: 6, SEQ ID No: 7, SEQ ID No: 8 and SEQ ID No: 9 and SEQ ID No: 10, preferably SEQ ID No. 9 and 10.

The present invention, according to a further aspect, relates to use of a marker for establishing the presence of a TSWV resistance gene or resistance conferring genomic sequence according to claim 8 or 9 in a tomato plant, wherein the marker is one or more selected from the group consisting of SEQ ID No: 5, SEQ ID No: 6, SEQ ID No: 7, SEQ ID No: 8, SEQ ID No: 9 and SEQ ID No: 10, preferably SEQ ID No. 9 and 10.

The present invention, according to a further aspect, relates to a method for providing a tomato plant that is resistant to a Tospovirus as described above, wherein the method comprises the steps of introducing a TSWV resistance gene that encodes for a TSWV resistance protein in the genome of a susceptible tomato plant, wherein the protein has at least 90% amino acid sequence identity with SEQ ID No.1.

According to a preferred embodiment, the present invention relates to the method method for providing a tomato plant that is resistant to a Tospovirus, wherein the step of introducing a TSWV resistance gene is achieved by genome editing techniques, for example CRISPR Cas. The present resistance gene, or genomic fragments, completely or in parts later to be reassembled, can inserted in the genome via CRISPR technology.

The present invention will be further detailed in the following figures and examples;

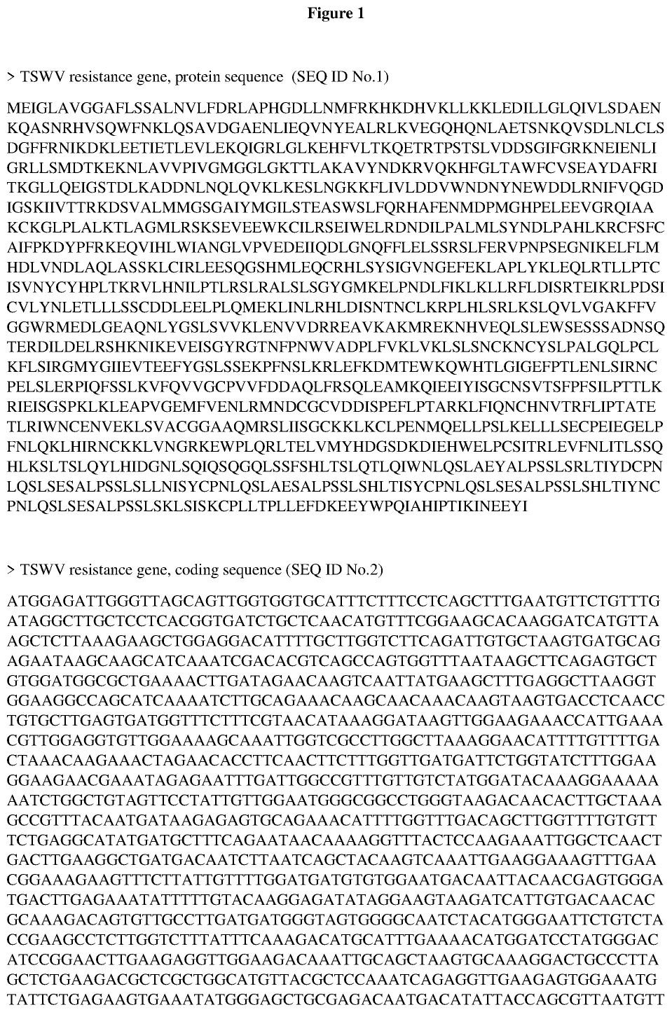

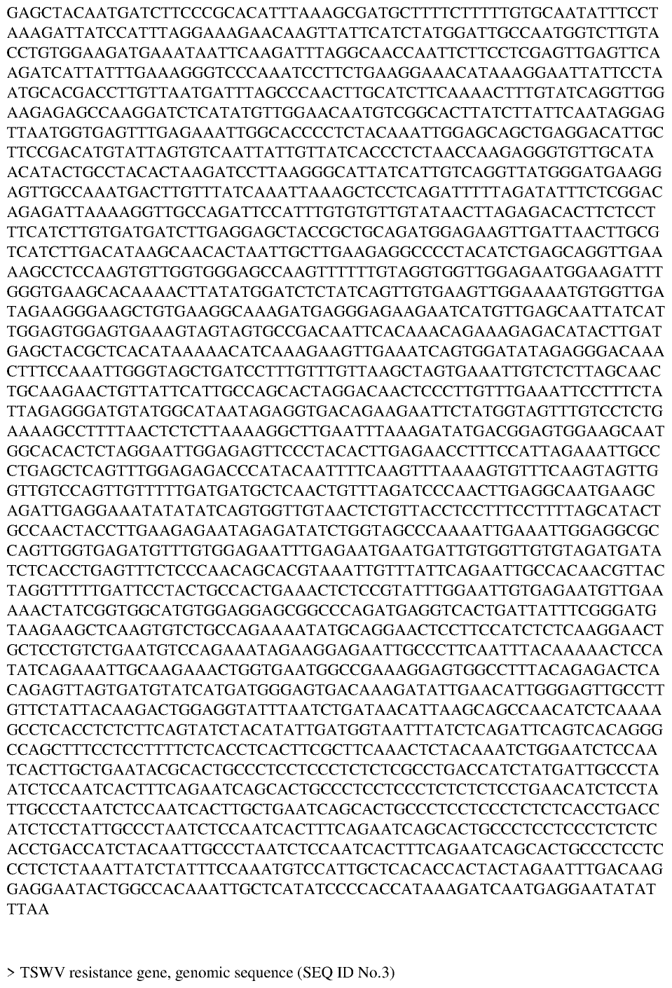

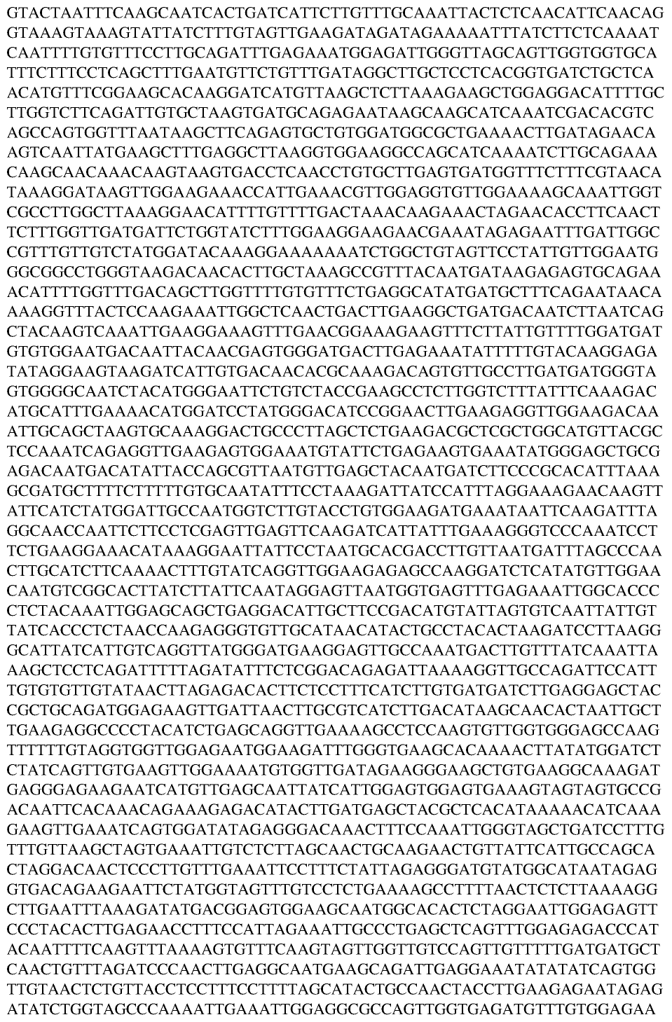

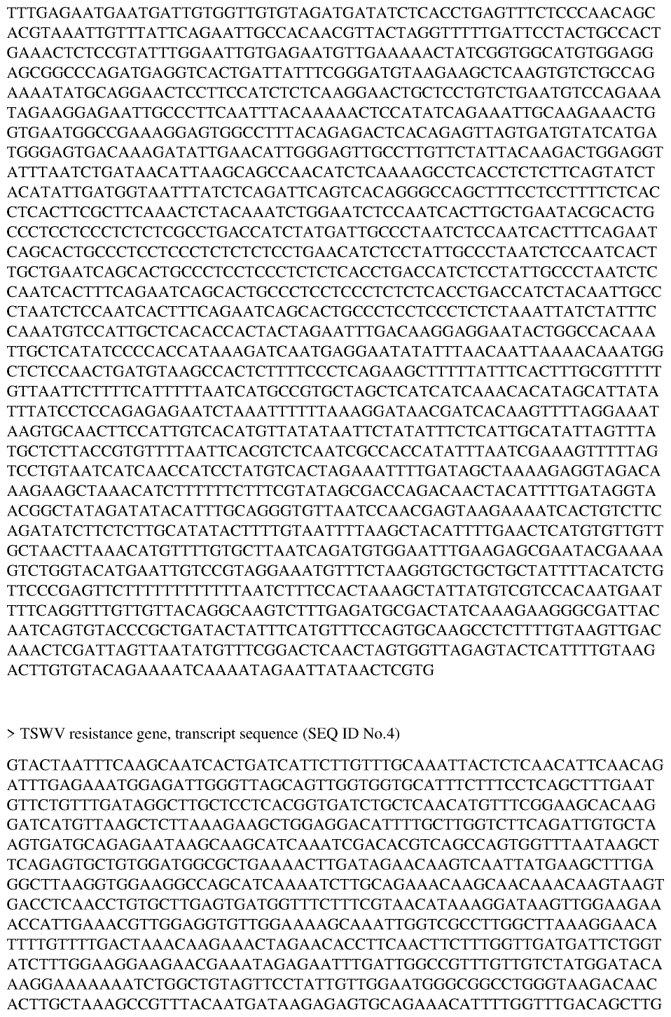

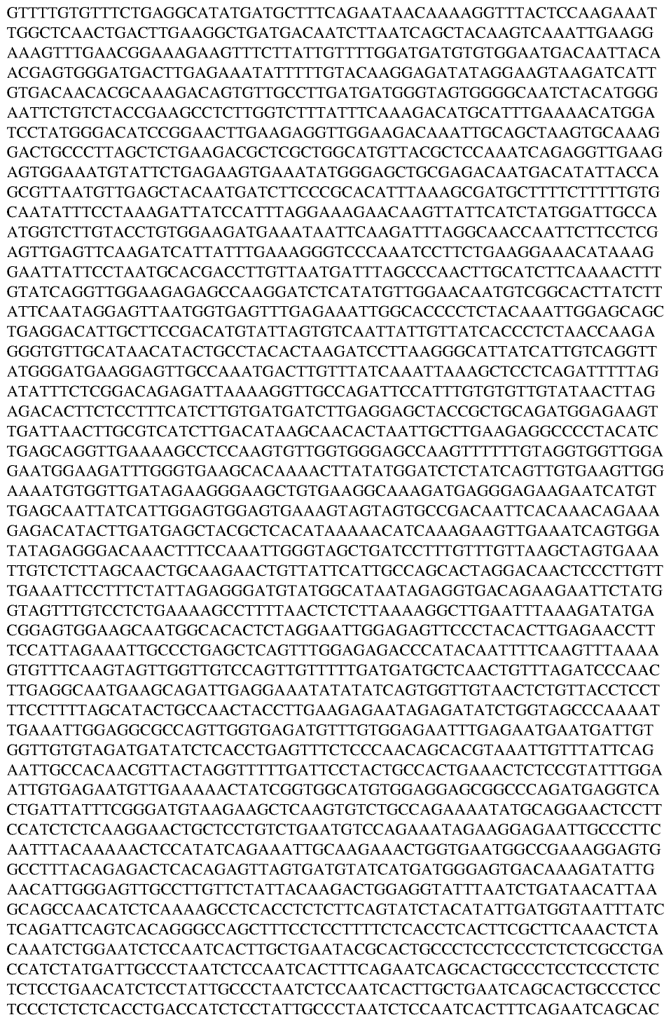

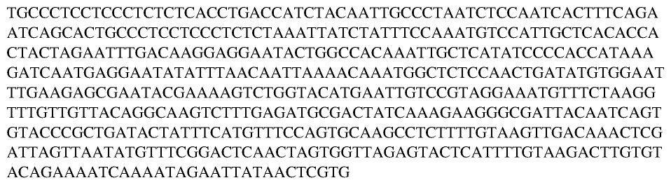

FIG. 1: Shows the protein sequence (SEQ ID No. 1), coding sequence (SEQ ID No. 2) and genomic sequence (SEQ ID No. 3), including a transcript sequence (SEQ ID No. 4, coding sequence including UTR sequence) of the TSWV resistance gene of present invention.

### EXAMPLES

Inoculation of a Tomato Plant with TSWV, Identifying Resistance.

The TSWV isolate D191 (Sw-5 breaking isolate, Australia) was used to perform the disease assays. As plant material, the Line OT9, which is a tomato inbred line susceptible for TSWV was used for virus maintenance. Symptomatic leaves received from the original samples were used for sap-mechanical inoculation on the Line OT9. The virus was maintained on systemically infected tomato plants OT9 by monthly sap-mechanical inoculation on new 3 weeks-old seedlings.

The tomato plants (S. peruvianum) have been screened. Seeds were sown in vermiculite, seedlings were transplanted in rockwool blocks and inoculated at 4 weeks after sowing. Plants were inoculated by mechanical infection with virus suspension in the fourth leaf stadium. Plants are scored for visual symptoms. The presence of small brownish ringspots on leaves, leaf deformation (purpling and upward rolling of leaves and stunting of leaves) and wilting of the plant was monitored. Plants were categorized as “resistant” when no such symptoms on leaves and the plant were observed. Plants displaying any of the symptoms were categorized as “susceptible”. Leaf samples were collected from asymptomatic plants (i.e resistant) to test for the presence of virus by ELISA. Resistant plants were selected from this test. Plant line OT1286 (comprising the Sw-5 resistance gene) and the susceptible plant line OT9 were used as controls.

The screening allowed the selection of several candidates for resistance breeding. The cuttings from the resistant F1 plants were used to produce F2 seeds. As these F1 plants were self-incompatible, F2 seeds were obtained by making crossings with different F1 plants (in the same background) and the F2 seeds were tested with isolate D191 and showed a monogenic incomplete dominant inheritance.

### Determination of TSWV Infection by ELISA

Infection was determined by ELISA. One apical leaf (fully expanded) of every plant was collected. Leaves were crushed using a Type R302 D63N-472 machine (VECTOR aandrijftechniek B.V., Rotterdam, The Netherlands) and sap was collected by adding 2 mL of PBS-Tween buffer. 100 μL of the extract was used for ELISA with antibodies against TSWV (supplier Prime Diagnostics, Wageningen, The Netherlands). ELISA reading was done by measurement of absorption at 405 nm with a FLUOstar Galaxy apparatus. Plants that gave absorption values more than 1.5 times of the clean control plants were considered infected (susceptible).

### Bioassays and Mapping of TSWV Resistance Genomic Sequence

The F2 population comprising 276 plants was tested in a bioassay with the TSWV isolate D191. Plants were phenotyped by eye (as described above) and resistant plants measured by Elisa. All plants were kept for F3 seed production. The plants that produced F3 seeds were tested in the D191 bioassay. Based on these results, a selection of 94 F2 plants was made for DNA genotyping with 96 markers distributed over the tomato genome. Markers located on the end of chromosome 11 were linked with the resistant phenotype.

Next, a segregating population of ˜1600 plants were genotyped with flanking markers M1 (position 51044245 bp on Chr 11) and M2 (position 52849899 bp) in order to select recombinant plants for fine mapping using TSWV No. AJ137 isolate. Next, 25 markers between M1 and M2 were developed for fine mapping of the resistance and the resistance was further fine mapped between position 52333713 bp and 52385754 bp. The selected plants after fine mapping were kept for F3 seed production for further fine mapping, which resulted in a region between 52373639 bp and 52385754 bp based on marker M18 and M22, which is a region of 12.114 bp based on the reference genome SL2.40 (see Table 1). Based on the reference genome SL2.40 and in silico prediction analysis (ITAG 2.3), one gene Solyc11g072000.1 is present in the fine mapped region that encodes for an NBS-LRR resistance protein.

Recombinant plants have been further genotyped with an additional in gene marker M27 (Table 1). This marker is located in the gene transcript of newly identified the TSWV resistance locus and is 100% linked with the TSWV resistance (see also Table 2).

TABLE 1

| Primer | | Pos. SL2.40, | SEQ ID |
|---|---|---|---|
| name | Primer sequence | Chr 11. | No. |
| M18_F | CAATGATAAATCGTGGTTGGT | 52373639 | 5 |
| M18_R | GTCATCATATTTACCAATCAATCAC | 52373730 | 6 |
| M22_F | CTTGTGATGAATGTTGAGATGTAC | 52385659 | 7 |
| M22_R | GTCAACTATCAATACTGCTTTTATC | 52385754 | 8 |
| M27_F | AAAGCCTTTTAACTCTCTTAAAAG | | 9 |
| M27_R | CTCAGGGCAATTTCTAATGG | | 10 |

### Identifying and Sequencing of the TSWV Resistance Locus in TSWV Resistant Plants (LYC00346)

Genomic DNA was isolated from a resistant plant (LYC00346, S. lycopersicum) of present invention, i.e. comprising the TSWV resistance locus, according to the protocol as published on 27 Apr. 2018 in Nature, Protocol Exchange (2018), Rachael Workman et al. “High Molecular Weight DNA Extraction from Recalcitrant Plant Species for Third Generation Sequencing”. The sequencing libraries were prepared using the PCR free, no multiplex, DNA Ligation Sequencing Kit-Promethion (SQK-LSK109). The isolation procedure resulted in high quality sequencing libraries to be used in the Oxford Nanopore system for sequencing (ONT sequencing). Promethion Flowcell Packs (3000 pore/flowcell) version R9.4.1. were used for sequencing.

Furthermore, to further resolve the TSWV locus and identify the gene providing the TSWV resistance, we performed ONT sequencing on the resistant plant. Sequencing of the entire transcript isoforms of the resistant plant was done using the Iso-Seq analysis application (Pacific Biosciences of California, PacBio).

Sequencing the resistant LYC00346 region using Oxford Nanopore sequencing technology resulted in a locus of 32,928 bp. Based on the fine mapping, the size and location of the genomic sequence that is harbouring the TSWV resistance gene, indicated as Sw-8 locus, between markers M18 and M22 is 20,814 bp larger compared to the SL2.40 reference genome of S. lycopersicum (12,114 bp vs. 32,928 bp, respectively). It is therefore highly likely that one or more genes are located within this region, providing the TSWV resistance.

Based on the reference genome SL2.40 and in silico prediction analysis (ITAG 2.3), at least one gene is located in the fine mapped region that encodes for a CC-NBS-LRR resistance protein, and is indicated as SEQ ID No.1 in this application.

### Validation TSWV Strain Resistance in Plant Comprising the TSWV Resistance Locus Comprising the Sw-8 Resistance Gene

Two resistance breaking mutations have been described in TSWV, both leading to amino acid changes in the non-structural movement (NSm) protein: C118Y and T120N. These amino acids are located in the 21-amino acid region of the NSm protein that is recognized by the Sw-5 protein (Zhu et al., 2017). Increased cultivation of Sw-5 varieties has recently led to the emergence of resistance breaking strains of TSWV in California US, and it was determined that these strains all contained the C118Y mutation (Batuman et al., 2018). Our diagnostics pipeline has identified several resistance breaking isolates of TSWV in samples from US (AJ137), and Italy (AJ034) and sequence analysis of the NSm gene showed that these breaking isolates had a mutation of amino acid 118, which is described as a determinant of breaking isolates; the identified strains carry a mutation of amino acid C118Y.

We confirmed the resistance-breaking nature of these isolates by bioassays with Sw-5 germplasm, whilst germplasm containing the Sw-8 locus was shown to be resistant to these isolates. Several tomato plants of present invention (S. lycopersicum) comprising the Sw-8 locus were tested for resistance against these TSWV isolates AJ034 (Italy), and AJ137 (US). The presence of the Sw-8 locus was determined by marker M27 (Table 2). As a control, plants were selected that did not contain the TSWV resistance locus, OT9. Table 2 shows the results obtained with the AJ034 and AJ137 isolates.

TABLE 2

| Plants | # | # | |
|---|---|---|---|
| (5 plants each) | Susceptible | Resistant | M27 |
| 21NSPL.0604 | | 5 | + |
| 21NSPL.0607 | 5 | | − |
| 21NSPL.0612 | | 5 | + |
| 21NSPL.0614 | | 5 | + |
| R line 0511-003 | | 5 | + |
| V line OT9 | 5 | | − |

The plants of present inventions, i.e. in case at least the marker M27 was present, showed to be resistant to the AJ034 and AJ137 isolates, indicating that the plant of present invention is resistant to the C118Y breaking TSWV isolates.

### Transcript Analysis, Identification of the TSWV Resistance Gene

To further resolve the TSWV resistance locus and identify the TSWV resistance gene providing the Sw-8 resistance, we sequenced the entire transcript isoforms of the resistant LYC00346 line using the Iso-Seq analysis application (Pacific Biosciences of California, PacBio). This resulted in three candidate transcripts which are located respectively at 3991-9274 bp (transcript 1), 10.393-20.755 bp (transcript 2) and 26.332-31.426 bp (transcript 3) at the Sw-8 resistance locus.

The transcript sequences have been examined on gene homology using public database of the National Center for Biotechnology Information (NCBI). All three transcript sequences have homology with the sequences that encode for NBS-LRR resistance proteins.

### Gene Validation Using VIGS

We further examined the role of the identified transcripts providing TSWV resistance and tried to identify if one or more of these transcripts is indeed the gene conferring resistance to TSWV. Therefore, a Virus Induced Gene Silencing (VIGS) analysis was performed. Tobacco rattle virus (TRV)-derived VIGS vectors have been abundantly described to study gene function in plants such as Arabidopsis thaliana, Nicotiana benthamiana, Solanum lycopersicum and other plants (see for example Huang C, Qian Y, Li Z, Zhou X.: Virus-induced gene silencing and its application in plant functional genomics. Sci China Life Sci. 2012; 55 (2): 99-108).

As such, three VIGS constructs were developed (Table 3, VIGS-1, VIGS-2 and VIGS-3), wherein each construct VIGS-1, 2, or 3 specifically targets transcript 1, 2, or transcript 3, respectively.

TABLE 3

| VIGS construct | Sequence |
|---|---|
| VIGS-1 (SEQ ID | GCCCACATAAAAACATTAAAGAAGTCAAAATCAGTGGATATAGA |
| No. 13) | GGGACAAACTTCCCCAATTGGGTAGCTGATCCTCTGTTTCTTAAG |
| | CTAGTGAAATTGTCTCTTAAAAACTGCAAGAACTGTTATTCCTTGC |
| | CAGCACTAGGACAACTCCCTTGTTTGAAATTCCTTTCTATTAGAGG |
| | GATGTATGGAATAAGAGTGGTGACGGAAGAATTCTATGGGAGAT |
| | TGTCCTCCAAAAAGCCCTTTAACTGTCTAGAGAAGCTTGAATTTG |
| | AAGATATGACGGAGTGGAAGCAATGGCACG |
| VIGS-2 (SEQ ID | GGGGATGTATGGCATAATAGAGGTGAAGGAAGAATTCTATGGTA |
| No. 14) | GTTTGTCCTCCGAAAAGCCTTTTAACTCTCTTGTGGAGCTTAGATT |
| | TAAAGATATGCCTGAGTGGAAGCAATGGCACACACTAGGAATTG |
| | GAGAGTTCCCTACACTTGAGAACCTTTTAATAGAAAATTGCCCTG |
| | ATCTCAGTTTGGAGACACCCATCCAATTTTCAAGTTTAAAAAGGT |
| | TTAGAGTCGTTGGTTGTCCAGTTGTTTTTGGTGATGCTCAGCTGTT |
| | TAGATCCCAACTTGAAGCAATGAAGCAGATTG |
| VIGS-3 (SEQ ID | CTATGGTAGTTTGTCCTCTGAAAAGCCTTTTAACTCTCTTAAAAGG |
| No. 15) | CTTGAATTTAAAGATATGACGGAGTGGAAGCAATGGCACACTCTA |
| | GGAATTGGAGAGTTCCCTACACTTGAGAACCTTTCCATTAGAAAT |
| | TGCCCTGAGCTCAGTTTGGAGAGACCCATACAATTTTCAAGTTTA |
| | AAAGTGTTTCAAGTAGTTGGTTGTCCAGTTGTTTTTGATGATGCTC |
| | AACTGTTTAGATCCCAACTTGAGGCAATGAAGCAGATTGAGGAA |
| | ATATATATCAGTGGTTGTAACTCTGTTACC |

The VIGS fragments were synthesized (IDT, gBlocks) and subsequently cloned into a TRV vector. The DNA sequences were confirmed by Sanger sequencing. The vector contains all sequences encoding for proteins that are required for a functional TRV particles including the target sequences. The VIGS vectors including the VIGS constructs were transformed into Agrobacterium tumefaciens strain GV3101 and used in VIGS experiments to reduce endogenous mRNA levels in tomato plants used in this experiment. A homozygous Sw-8 resistant line (0511-003) of present invention, an Sw-5 resistance gene comprising line (OT1286) and a susceptible control line (OT9) were used in the VIGS experiment, in which plants were Agrobacterium infiltrated at seedling stage (cotyledons) followed by TSWV inoculation three weeks after Agrobacterium infiltration. Two weeks after TSWV inoculation the individual plants were phenotyped by ELISA.

The OT9 and the Sw-5 plants were susceptible, as expected. The Sw-8 resistant line 0511-003, which was shown earlier to be fully resistant, became susceptible to TSWV in case the transcript 3 was silenced using the VIGS-3 construct designed to specifically target this gene, whereas silencing using the VIGS-1 and 2 constructs did not result in any susceptibility of the plants tested. Based on these results it can be concluded that gene 3 is the conferring resistance gene to TSWV. Susceptibility was found in resistant plants infiltrated with construct VIGS-3, whereas nearly no susceptibility has been detected in resistant plants infiltrated using constructs VIGS-1 and 2 (Table 4). Based on these results it can be concluded that the coding sequence of transcript 3 (included herein as SEQ ID No. 4) is the conferring resistance to TSWV.

TABLE 4

| | # | VIGS | TSWV | # S | # R |
|---|---|---|---|---|---|
| Plants | plants | construct | infection | plants | plants |
| R line 0511-003 | 18 | VIGS-1 | Yes | 0 | 18 |
| R line 0511-003 | 18 | VIGS-2 | Yes | 2 | 16 |
| R line 0511-003 | 18 | VIGS-3 | Yes | 17 | 1 |
| R line 0511-003 | 6 | No | Yes | 0 | 6 |
| S line OT1286 | 6 | No | Yes | 6 | 0 |
| S line OT9 | 6 | No | Yes | 6 | 0 |

Targeted Insertion of Sw-8 Gene in S. lycopersicum by CRISPR/Cas9

By using the CRISPR/Cas9 system, double stranded breaks (DSB) can be introduced into the genome of the tomato plant. These DSB can be repaired by 2 mechanisms, non-homologous end-joining (NHEJ) or homology directed repair (HDR). HDR relies on the presence of a donor repair template (DRT) in the vicinity of the DSB. It has been shown for several crops that the low efficiency of HDR can be improved by using Geminivirus replicons (GVR) to provide enough donor repair template into the cell, including tobacco, and tomato (Baltes et al. 2014; Cermak et al. 2015; Dahan-Meir et al 2018). Using this system and in combination with the replicon of the BYDV (bean yellow dwarf virus) enables to achieve targeted integration of DNA into the genome, i.e. in the target region on chromosome 11 of a tomato plant (S. lycopersicum). The donor sequence was flanked on both sides with homology arms of 250 bp, matching the sequences flanking the DSB, to facilitate directional insertion.

Plants were transformed with one construct containing all components for the gene insertion strategy. In short regarding this construct, according to Dahan-Meir et al. with minor modifications, the Cas9 under the Ubi10 promoter, a gRNA targeting the location of the fine mapping in S. lycopersicum on chromosome 11 driven by the U6-26 promoter. The sequence of the gRNA is ACCAAGTATAACCCAACAAGTGG (PAM sequence underlined) and is in the intergenic region just next to Solyc11g072000.1. This plasmid further contains the viral rolling circle replication (RCR) components, the replication initiator complex (Rep and RepA), and the large and small intergenic region (LIR, SIR) all described in Dahan-Meir et al., Via this experimental setup, strong Cas9 expression, and efficient amplification of the donor template was achieved since all components are present on one construct. Within the replicon sequence, the Sw-8 sequence was integrated. To obtain proper expression, we added the 500 bp before the startcodon that includes the UTR and promoter region. The coding sequence consisted of 3966 bp (SEQ ID No. 2) combined with the 500 bp, and the two 250 bp homology arms, generating a total of 4966 bp template was included in the replicon.

Next, tomato cotyledon transformation using Agrobacterium was performed essentially as described by Van Eck et al. (2018). To verify the integration of the Sw-8 gene, specific PCR was performed using primers that will only yield an amplicon on the successfully repaired HDR-event; one primer annealing to Sw-8, and one primer annealing to the insertion location just outside of the 250 bp homology arm to prevent false positives from amplifying the non-integrated donor.

Amplicons were subsequently Sanger sequenced to confirm correct insertion. Plants containing the Sw-8 gene were set for seeds and the T1 progeny was genotyped again to confirm the heritability. The plants were put in an TSWV test and scored for disease symptoms. The expected resistant phenotype was observed an no TSWV disease symptoms were observed in the plants comprising the Sw-8 gene.

1. A plant of the S. lycopersicum species that is resistant to a Tospovirus, wherein the plant comprises a TSWV resistance gene that encodes for a TSWV resistance protein, wherein the TSWV resistance protein has at least 90% amino acid sequence identity with SEQ ID NO: 1.

2. The plant according to claim 1, wherein the TSWV resistance gene comprises a coding sequence that has at least 90% sequence identity with SEQ ID NO: 2 and/or SEQ ID NO: 4.

3. The plant according to claim 1, wherein the plant comprises a genomic sequence having at least 90% sequence identity with SEQ ID NO: 3.

4. The plant according to claim 1, wherein the Tospovirus is one or more selected from the group consisting of Tomato spotted wilt virus (TSWV), Groundnut ringspot virus (GRSV), Groundnut bud necrosis virus (GBNV), Capsicum chlorosis virus (CaCV), and Tomato chlorotic spot virus (TCSV), preferably TSWV.

5. The plant according to claim 4, wherein the TSWV comprises a C118Y mutation and/or T120N mutation in a non-structural movement (NSm) protein of TSWV.

6. The plant according to claim 1, wherein the TSWV resistance gene is homozygous present in a genome of said plant.

7. The plant according to claim 1, wherein the TSWV resistance gene is as found in the deposit accession number NCIMB 43771.

8. The plant according to claim 1, wherein the plant further comprises an Sw-5 resistance gene that encodes for Sw-5 protein having at least 95% amino acid sequence identity with SEQ ID NO: 11.

9. The plant according to claim 1, wherein the TSWV resistance protein is of Solanum peruvianum.

10. A plant, plant part, tissue, cell, or seed derived from the plant according to claim 1.

11. A resistance gene for providing resistance to a Tospovirus in a S. lycopersicum plant, wherein said resistance gene is represented by a coding sequence having at least 90% sequence identity with SEQ ID NO: 2 and/or SEQ ID NO: 4.

12. A genomic sequence for providing resistance to a Tospovirus in a S. lycopersicum plant, wherein the genomic sequence has at least 90% sequence identity with SEQ ID NO: 3.

13. The resistance gene according to claim 11 or the genomic sequence according to claim 12, wherein the Tospovirus is one or more selected from the group consisting of Tomato spotted wilt virus (TSWV), Groundnut ringspot virus (GRSV), Groundnut bud necrosis virus (GBNV), Capsicum chlorosis virus (CaCV), and Tomato chlorotic spot virus (TCSV), preferably TSWV.

14. The resistance gene or the genomic sequence according to claim 13, wherein the TSWV comprises a C118Y mutation and/or T120N mutation in a non-structural movement (NSm) protein of TSWV.

15. A method for providing a plant of the S. lycopersicum species that is resistant to Tospovirus, wherein the method comprises the steps of:
a) selecting a tomato plant that is resistant to Tospovirus, wherein said selection comprises establishing the presence of the resistance gene according to claim 11 or the genomic sequence according to claim 12, preferably said resistance gene, preferably wherein said tomato plant is an S. lycopersicum plant or an S. peruvianum plant;
b) transferring, for example by crossing, the identified resistance gene, genomic sequence or locus of step a) into a S. lycopersicum plant thereby conferring Tospovirus resistance to said S. lycopersicum plant.

16. (canceled)

17. The method according to claim 15, wherein after step b), the method comprises selecting and crossing a first S. lycopersicum plant that is resistant to Tospovirus with a second S. lycopersicum plant that is not resistant to Tospovirus, and subsequently selecting a S. lycopersicum plant that is resistant to Tospovirus.

18. The method according to claim 15, wherein in step a) establishing the presence of the resistance gene or the genomic sequence in the tomato plant is performed by one or more markers selected from the group consisting of SEQ ID NO: 5, SEQ ID NO: 6, SEQ ID NO: 7, SEQ ID NO: 8 and SEQ ID NO: 9 and SEQ ID NO: 10, preferably SEQ ID NOS: 9 and 10.

19. A method for providing the plant of S. lycopersicum species that is resistant to the Tospovirus according to claim 1, wherein the method comprises introducing the TSWV resistance gene that encodes for the TSWV resistance protein into a genome of a susceptible tomato plant, wherein the TSWV resistance protein has at least 90% amino acid sequence identity with SEQ ID NO: 1.

20. The method according to claim 19, wherein the step of introducing the TSWV resistance gene is achieved by genome editing techniques comprising CRISPR Cas.

21. A method, comprising using a marker for establishing presence of the resistance gene according to claim 11 or the genomic sequence according to claim 12 in a tomato plant, wherein the marker is one or more selected from the group consisting of SEQ ID NO: 5, SEQ ID NO: 6, SEQ ID NO: 7, SEQ ID NO: 8, and SEQ ID NO: 9 and SEQ ID NO: 10 preferably SEQ ID NOS: 9 and 10.


SEQUENCE LISTING (15 sequences)

SEQ ID NO: 1 - protein, 1321 aa, Solanum peruvianum
  TSWV resistance protein sequence
MEIGLAVGGAFLSSALNVLFDRLAPHGDLLNMFRKHKDHVKLLKKLEDILLGLQIVLSDAENKQASNRHVSQWFNKLQSAVDGAENLIEQVNYEALRLKVEGQHQNLAETSNKQVSDLNLCLSDGFFRNIKDKLEETIETLEVLEKQIGRLGLKEHFVLTKQETRTPSTSLVDDSGIFGRKNEIENLIGRLLSMDTKEKNLAVVPIVGMGGLGKTTLAKAVYNDKRVQKHFGLTAWFCVSEAYDAFRITKGLLQEIGSTDLKADDNLNQLQVKLKESLNGKKFLIVLDDVWNDNYNEWDDLRNIFVQGDIGSKIIVTTRKDSVALMMGSGAIYMGILSTEASWSLFQRHAFENMDPMGHPELEEVGRQIAAKCKGLPLALKTLAGMLRSKSEVEEWKCILRSEIWELRDNDILPALMLSYNDLPAHLKRCFSFCAIFPKDYPFRKEQVIHLWIANGLVPVEDEIIQDLGNQFFLELSSRSLFERVPNPSEGNIKELFLMHDLVNDLAQLASSKLCIRLEESQGSHMLEQCRHLSYSIGVNGEFEKLAPLYKLEQLRTLLPTCISVNYCYHPLTKRVLHNILPTLRSLRALSLSGYGMKELPNDLFIKLKLLRFLDISRTEIKRLPDSICVLYNLETLLLSSCDDLEELPLQMEKLINLRHLDISNTNCLKRPLHLSRLKSLQVLVGAKFFVGGWRMEDLGEAQNLYGSLSVVKLENVVDRREAVKAKMREKNHVEQLSLEWSESSSADNSQTERDILDELRSHKNIKEVEISGYRGTNFPNWVADPLFVKLVKLSLSNCKNCYSLPALGQLPCLKFLSIRGMYGIIEVTEEFYGSLSSEKPFNSLKRLEFKDMTEWKQWHTLGIGEFPTLENLSIRNCPELSLERPIQFSSLKVFQVVGCPVVFDDAQLFRSQLEAMKQIEEIYISGCNSVTSFPFSILPTTLKRIEISGSPKLKLEAPVGEMFVENLRMNDCGCVDDISPEFLPTARKLFIQNCHNVTRFLIPTATETLRIWNCENVEKLSVACGGAAQMRSLIISGCKKLKCLPENMQELLPSLKELLLSECPEIEGELPFNLQKLHIRNCKKLVNGRKEWPLQRLTELVMYHDGSDKDIEHWELPCSITRLEVFNLITLSSQHLKSLTSLQYLHIDGNLSQIQSQGQLSSFSHLTSLQTLQIWNLQSLAEYALPSSLSRLTIYDCPNLQSLSESALPSSLSLLNISYCPNLQSLAESALPSSLSHLTISYCPNLQSLSESALPSSLSHLTIYNCPNLQSLSESALPSSLSKLSISKCPLLTPLLEFDKEEYWPQIAHIPTIKINEEYI

SEQ ID NO: 2 - DNA, 3966 bp, Solanum peruvianum
  cDNA
atggagattgggttagcagttggtggtgcatttctttcctcagctttgaatgttctgtttgataggcttgctcctcacggtgatctgctcaacatgtttcggaagcacaaggatcatgttaagctcttaaagaagctggaggacattttgcttggtcttcagattgtgctaagtgatgcagagaataagcaagcatcaaatcgacacgtcagccagtggtttaataagcttcagagtgctgtggatggcgctgaaaacttgatagaacaagtcaattatgaagctttgaggcttaaggtggaaggccagcatcaaaatcttgcagaaacaagcaacaaacaagtaagtgacctcaacctgtgcttgagtgatggtttctttcgtaacataaaggataagttggaagaaaccattgaaacgttggaggtgttggaaaagcaaattggtcgccttggcttaaaggaacattttgttttgactaaacaagaaactagaacaccttcaacttctttggttgatgattctggtatctttggaaggaagaacgaaatagagaatttgattggccgtttgttgtctatggatacaaaggaaaaaaatctggctgtagttcctattgttggaatgggcggcctgggtaagacaacacttgctaaagccgtttacaatgataagagagtgcagaaacattttggtttgacagcttggttttgtgtttctgaggcatatgatgctttcagaataacaaaaggtttactccaagaaattggctcaactgacttgaaggctgatgacaatcttaatcagctacaagtcaaattgaaggaaagtttgaacggaaagaagtttcttattgttttggatgatgtgtggaatgacaattacaacgagtgggatgacttgagaaatatttttgtacaaggagatataggaagtaagatcattgtgacaacacgcaaagacagtgttgccttgatgatgggtagtggggcaatctacatgggaattctgtctaccgaagcctcttggtctttatttcaaagacatgcatttgaaaacatggatcctatgggacatccggaacttgaagaggttggaagacaaattgcagctaagtgcaaaggactgcccttagctctgaagacgctcgctggcatgttacgctccaaatcagaggttgaagagtggaaatgtattctgagaagtgaaatatgggagctgcgagacaatgacatattaccagcgttaatgttgagctacaatgatcttcccgcacatttaaagcgatgcttttctttttgtgcaatatttcctaaagattatccatttaggaaagaacaagttattcatctatggattgccaatggtcttgtacctgtggaagatgaaataattcaagatttaggcaaccaattcttcctcgagttgagttcaagatcattatttgaaagggtcccaaatccttctgaaggaaacataaaggaattattcctaatgcacgaccttgttaatgatttagcccaacttgcatcttcaaaactttgtatcaggttggaagagagccaaggatctcatatgttggaacaatgtcggcacttatcttattcaataggagttaatggtgagtttgagaaattggcacccctctacaaattggagcagctgaggacattgcttccgacatgtattagtgtcaattattgttatcaccctctaaccaagagggtgttgcataacatactgcctacactaagatccttaagggcattatcattgtcaggttatgggatgaaggagttgccaaatgacttgtttatcaaattaaagctcctcagatttttagatatttctcggacagagattaaaaggttgccagattccatttgtgtgttgtataacttagagacacttctcctttcatcttgtgatgatcttgaggagctaccgctgcagatggagaagttgattaacttgcgtcatcttgacataagcaacactaattgcttgaagaggcccctacatctgagcaggttgaaaagcctccaagtgttggtgggagccaagttttttgtaggtggttggagaatggaagatttgggtgaagcacaaaacttatatggatctctatcagttgtgaagttggaaaatgtggttgatagaagggaagctgtgaaggcaaagatgagggagaagaatcatgttgagcaattatcattggagtggagtgaaagtagtagtgccgacaattcacaaacagaaagagacatacttgatgagctacgctcacataaaaacatcaaagaagttgaaatcagtggatatagagggacaaactttccaaattgggtagctgatcctttgtttgttaagctagtgaaattgtctcttagcaactgcaagaactgttattcattgccagcactaggacaactcccttgtttgaaattcctttctattagagggatgtatggcataatagaggtgacagaagaattctatggtagtttgtcctctgaaaagccttttaactctcttaaaaggcttgaatttaaagatatgacggagtggaagcaatggcacactctaggaattggagagttccctacacttgagaacctttccattagaaattgccctgagctcagtttggagagacccatacaattttcaagtttaaaagtgtttcaagtagttggttgtccagttgtttttgatgatgctcaactgtttagatcccaacttgaggcaatgaagcagattgaggaaatatatatcagtggttgtaactctgttacctcctttccttttagcatactgccaactaccttgaagagaatagagatatctggtagcccaaaattgaaattggaggcgccagttggtgagatgtttgtggagaatttgagaatgaatgattgtggttgtgtagatgatatctcacctgagtttctcccaacagcacgtaaattgtttattcagaattgccacaacgttactaggtttttgattcctactgccactgaaactctccgtatttggaattgtgagaatgttgaaaaactatcggtggcatgtggaggagcggcccagatgaggtcactgattatttcgggatgtaagaagctcaagtgtctgccagaaaatatgcaggaactccttccatctctcaaggaactgctcctgtctgaatgtccagaaatagaaggagaattgcccttcaatttacaaaaactccatatcagaaattgcaagaaactggtgaatggccgaaaggagtggcctttacagagactcacagagttagtgatgtatcatgatgggagtgacaaagatattgaacattgggagttgccttgttctattacaagactggaggtatttaatctgataacattaagcagccaacatctcaaaagcctcacctctcttcagtatctacatattgatggtaatttatctcagattcagtcacagggccagctttcctccttttctcacctcacttcgcttcaaactctacaaatctggaatctccaatcacttgctgaatacgcactgccctcctccctctctcgcctgaccatctatgattgccctaatctccaatcactttcagaatcagcactgccctcctccctctctctcctgaacatctcctattgccctaatctccaatcacttgctgaatcagcactgccctcctccctctctcacctgaccatctcctattgccctaatctccaatcactttcagaatcagcactgccctcctccctctctcacctgaccatctacaattgccctaatctccaatcactttcagaatcagcactgccctcctccctctctaaattatctatttccaaatgtccattgctcacaccactactagaatttgacaaggaggaatactggccacaaattgctcatatccccaccataaagatcaatgaggaatatatttaa

SEQ ID NO: 3 - DNA, 5083 bp, Solanum peruvianum
  gDNA
gtactaatttcaagcaatcactgatcattcttgtttgcaaattactctcaacattcaacaggtaaagtaaagtattatctttgtagttgaagatagatagaaaaatttatcttctcaaaatcaattttgtgtttccttgcagatttgagaaatggagattgggttagcagttggtggtgcatttctttcctcagctttgaatgttctgtttgataggcttgctcctcacggtgatctgctcaacatgtttcggaagcacaaggatcatgttaagctcttaaagaagctggaggacattttgcttggtcttcagattgtgctaagtgatgcagagaataagcaagcatcaaatcgacacgtcagccagtggtttaataagcttcagagtgctgtggatggcgctgaaaacttgatagaacaagtcaattatgaagctttgaggcttaaggtggaaggccagcatcaaaatcttgcagaaacaagcaacaaacaagtaagtgacctcaacctgtgcttgagtgatggtttctttcgtaacataaaggataagttggaagaaaccattgaaacgttggaggtgttggaaaagcaaattggtcgccttggcttaaaggaacattttgttttgactaaacaagaaactagaacaccttcaacttctttggttgatgattctggtatctttggaaggaagaacgaaatagagaatttgattggccgtttgttgtctatggatacaaaggaaaaaaatctggctgtagttcctattgttggaatgggcggcctgggtaagacaacacttgctaaagccgtttacaatgataagagagtgcagaaacattttggtttgacagcttggttttgtgtttctgaggcatatgatgctttcagaataacaaaaggtttactccaagaaattggctcaactgacttgaaggctgatgacaatcttaatcagctacaagtcaaattgaaggaaagtttgaacggaaagaagtttcttattgttttggatgatgtgtggaatgacaattacaacgagtgggatgacttgagaaatatttttgtacaaggagatataggaagtaagatcattgtgacaacacgcaaagacagtgttgccttgatgatgggtagtggggcaatctacatgggaattctgtctaccgaagcctcttggtctttatttcaaagacatgcatttgaaaacatggatcctatgggacatccggaacttgaagaggttggaagacaaattgcagctaagtgcaaaggactgcccttagctctgaagacgctcgctggcatgttacgctccaaatcagaggttgaagagtggaaatgtattctgagaagtgaaatatgggagctgcgagacaatgacatattaccagcgttaatgttgagctacaatgatcttcccgcacatttaaagcgatgcttttctttttgtgcaatatttcctaaagattatccatttaggaaagaacaagttattcatctatggattgccaatggtcttgtacctgtggaagatgaaataattcaagatttaggcaaccaattcttcctcgagttgagttcaagatcattatttgaaagggtcccaaatccttctgaaggaaacataaaggaattattcctaatgcacgaccttgttaatgatttagcccaacttgcatcttcaaaactttgtatcaggttggaagagagccaaggatctcatatgttggaacaatgtcggcacttatcttattcaataggagttaatggtgagtttgagaaattggcacccctctacaaattggagcagctgaggacattgcttccgacatgtattagtgtcaattattgttatcaccctctaaccaagagggtgttgcataacatactgcctacactaagatccttaagggcattatcattgtcaggttatgggatgaaggagttgccaaatgacttgtttatcaaattaaagctcctcagatttttagatatttctcggacagagattaaaaggttgccagattccatttgtgtgttgtataacttagagacacttctcctttcatcttgtgatgatcttgaggagctaccgctgcagatggagaagttgattaacttgcgtcatcttgacataagcaacactaattgcttgaagaggcccctacatctgagcaggttgaaaagcctccaagtgttggtgggagccaagttttttgtaggtggttggagaatggaagatttgggtgaagcacaaaacttatatggatctctatcagttgtgaagttggaaaatgtggttgatagaagggaagctgtgaaggcaaagatgagggagaagaatcatgttgagcaattatcattggagtggagtgaaagtagtagtgccgacaattcacaaacagaaagagacatacttgatgagctacgctcacataaaaacatcaaagaagttgaaatcagtggatatagagggacaaactttccaaattgggtagctgatcctttgtttgttaagctagtgaaattgtctcttagcaactgcaagaactgttattcattgccagcactaggacaactcccttgtttgaaattcctttctattagagggatgtatggcataatagaggtgacagaagaattctatggtagtttgtcctctgaaaagccttttaactctcttaaaaggcttgaatttaaagatatgacggagtggaagcaatggcacactctaggaattggagagttccctacacttgagaacctttccattagaaattgccctgagctcagtttggagagacccatacaattttcaagtttaaaagtgtttcaagtagttggttgtccagttgtttttgatgatgctcaactgtttagatcccaacttgaggcaatgaagcagattgaggaaatatatatcagtggttgtaactctgttacctcctttccttttagcatactgccaactaccttgaagagaatagagatatctggtagcccaaaattgaaattggaggcgccagttggtgagatgtttgtggagaatttgagaatgaatgattgtggttgtgtagatgatatctcacctgagtttctcccaacagcacgtaaattgtttattcagaattgccacaacgttactaggtttttgattcctactgccactgaaactctccgtatttggaattgtgagaatgttgaaaaactatcggtggcatgtggaggagcggcccagatgaggtcactgattatttcgggatgtaagaagctcaagtgtctgccagaaaatatgcaggaactccttccatctctcaaggaactgctcctgtctgaatgtccagaaatagaaggagaattgcccttcaatttacaaaaactccatatcagaaattgcaagaaactggtgaatggccgaaaggagtggcctttacagagactcacagagttagtgatgtatcatgatgggagtgacaaagatattgaacattgggagttgccttgttctattacaagactggaggtatttaatctgataacattaagcagccaacatctcaaaagcctcacctctcttcagtatctacatattgatggtaatttatctcagattcagtcacagggccagctttcctccttttctcacctcacttcgcttcaaactctacaaatctggaatctccaatcacttgctgaatacgcactgccctcctccctctctcgcctgaccatctatgattgccctaatctccaatcactttcagaatcagcactgccctcctccctctctctcctgaacatctcctattgccctaatctccaatcacttgctgaatcagcactgccctcctccctctctcacctgaccatctcctattgccctaatctccaatcactttcagaatcagcactgccctcctccctctctcacctgaccatctacaattgccctaatctccaatcactttcagaatcagcactgccctcctccctctctaaattatctatttccaaatgtccattgctcacaccactactagaatttgacaaggaggaatactggccacaaattgctcatatccccaccataaagatcaatgaggaatatatttaacaattaaaacaaatggctctccaactgatgtaagccactcttttccctcagaagctttttatttcactttgcgtttttgttaattcttttcatttttaatcatgccgtgctagctcatcatcaaacacatagcattatatttatcctccagagagaatctaaattttttaaaggataacgatcacaagttttaggaaataagtgcaacttccattgtcacatgttatataattctatatttctcattgcatattagtttatgctcttaccgtgttttaattcacgtctcaatcgccaccatatttaatcgaaagtttttagtcctgtaatcatcaaccatcctatgtcactagaaattttgatagctaaaagaggtagacaaagaagctaaacatcttttttctttcgtatagcgaccagacaactacattttgataggtaacggctatagatatacatttgcagggtgttaatccaacgagtaagaaaatcactgtcttcagatatcttctcttgcatatacttttgtaattttaagctacattttgaactcatgtgttgttgctaacttaaacatgttttgtgcttaatcagatgtggaatttgaagagcgaatacgaaaagtctggtacatgaattgtccgtaggaaatgtttctaaggtgctgctgctattttacatctgttcccgagttcttttttttttttaatctttccactaaagctattatgtcgtccacaatgaattttcaggtttgttgttacaggcaagtctttgagatgcgactatcaaagaagggcgattacaatcagtgtacccgctgatactatttcatgtttccagtgcaagcctcttttgtaagttgacaaactcgattagttaatatgtttcggactcaactagtggttagagtactcattttgtaagacttgtgtacagaaaatcaaaatagaattataactcgtg

SEQ ID NO: 4 - DNA, 4346 bp, Solanum peruvianum
  transcript 3
gtactaatttcaagcaatcactgatcattcttgtttgcaaattactctcaacattcaacagatttgagaaatggagattgggttagcagttggtggtgcatttctttcctcagctttgaatgttctgtttgataggcttgctcctcacggtgatctgctcaacatgtttcggaagcacaaggatcatgttaagctcttaaagaagctggaggacattttgcttggtcttcagattgtgctaagtgatgcagagaataagcaagcatcaaatcgacacgtcagccagtggtttaataagcttcagagtgctgtggatggcgctgaaaacttgatagaacaagtcaattatgaagctttgaggcttaaggtggaaggccagcatcaaaatcttgcagaaacaagcaacaaacaagtaagtgacctcaacctgtgcttgagtgatggtttctttcgtaacataaaggataagttggaagaaaccattgaaacgttggaggtgttggaaaagcaaattggtcgccttggcttaaaggaacattttgttttgactaaacaagaaactagaacaccttcaacttctttggttgatgattctggtatctttggaaggaagaacgaaatagagaatttgattggccgtttgttgtctatggatacaaaggaaaaaaatctggctgtagttcctattgttggaatgggcggcctgggtaagacaacacttgctaaagccgtttacaatgataagagagtgcagaaacattttggtttgacagcttggttttgtgtttctgaggcatatgatgctttcagaataacaaaaggtttactccaagaaattggctcaactgacttgaaggctgatgacaatcttaatcagctacaagtcaaattgaaggaaagtttgaacggaaagaagtttcttattgttttggatgatgtgtggaatgacaattacaacgagtgggatgacttgagaaatatttttgtacaaggagatataggaagtaagatcattgtgacaacacgcaaagacagtgttgccttgatgatgggtagtggggcaatctacatgggaattctgtctaccgaagcctcttggtctttatttcaaagacatgcatttgaaaacatggatcctatgggacatccggaacttgaagaggttggaagacaaattgcagctaagtgcaaaggactgcccttagctctgaagacgctcgctggcatgttacgctccaaatcagaggttgaagagtggaaatgtattctgagaagtgaaatatgggagctgcgagacaatgacatattaccagcgttaatgttgagctacaatgatcttcccgcacatttaaagcgatgcttttctttttgtgcaatatttcctaaagattatccatttaggaaagaacaagttattcatctatggattgccaatggtcttgtacctgtggaagatgaaataattcaagatttaggcaaccaattcttcctcgagttgagttcaagatcattatttgaaagggtcccaaatccttctgaaggaaacataaaggaattattcctaatgcacgaccttgttaatgatttagcccaacttgcatcttcaaaactttgtatcaggttggaagagagccaaggatctcatatgttggaacaatgtcggcacttatcttattcaataggagttaatggtgagtttgagaaattggcacccctctacaaattggagcagctgaggacattgcttccgacatgtattagtgtcaattattgttatcaccctctaaccaagagggtgttgcataacatactgcctacactaagatccttaagggcattatcattgtcaggttatgggatgaaggagttgccaaatgacttgtttatcaaattaaagctcctcagatttttagatatttctcggacagagattaaaaggttgccagattccatttgtgtgttgtataacttagagacacttctcctttcatcttgtgatgatcttgaggagctaccgctgcagatggagaagttgattaacttgcgtcatcttgacataagcaacactaattgcttgaagaggcccctacatctgagcaggttgaaaagcctccaagtgttggtgggagccaagttttttgtaggtggttggagaatggaagatttgggtgaagcacaaaacttatatggatctctatcagttgtgaagttggaaaatgtggttgatagaagggaagctgtgaaggcaaagatgagggagaagaatcatgttgagcaattatcattggagtggagtgaaagtagtagtgccgacaattcacaaacagaaagagacatacttgatgagctacgctcacataaaaacatcaaagaagttgaaatcagtggatatagagggacaaactttccaaattgggtagctgatcctttgtttgttaagctagtgaaattgtctcttagcaactgcaagaactgttattcattgccagcactaggacaactcccttgtttgaaattcctttctattagagggatgtatggcataatagaggtgacagaagaattctatggtagtttgtcctctgaaaagccttttaactctcttaaaaggcttgaatttaaagatatgacggagtggaagcaatggcacactctaggaattggagagttccctacacttgagaacctttccattagaaattgccctgagctcagtttggagagacccatacaattttcaagtttaaaagtgtttcaagtagttggttgtccagttgtttttgatgatgctcaactgtttagatcccaacttgaggcaatgaagcagattgaggaaatatatatcagtggttgtaactctgttacctcctttccttttagcatactgccaactaccttgaagagaatagagatatctggtagcccaaaattgaaattggaggcgccagttggtgagatgtttgtggagaatttgagaatgaatgattgtggttgtgtagatgatatctcacctgagtttctcccaacagcacgtaaattgtttattcagaattgccacaacgttactaggtttttgattcctactgccactgaaactctccgtatttggaattgtgagaatgttgaaaaactatcggtggcatgtggaggagcggcccagatgaggtcactgattatttcgggatgtaagaagctcaagtgtctgccagaaaatatgcaggaactccttccatctctcaaggaactgctcctgtctgaatgtccagaaatagaaggagaattgcccttcaatttacaaaaactccatatcagaaattgcaagaaactggtgaatggccgaaaggagtggcctttacagagactcacagagttagtgatgtatcatgatgggagtgacaaagatattgaacattgggagttgccttgttctattacaagactggaggtatttaatctgataacattaagcagccaacatctcaaaagcctcacctctcttcagtatctacatattgatggtaatttatctcagattcagtcacagggccagctttcctccttttctcacctcacttcgcttcaaactctacaaatctggaatctccaatcacttgctgaatacgcactgccctcctccctctctcgcctgaccatctatgattgccctaatctccaatcactttcagaatcagcactgccctcctccctctctctcctgaacatctcctattgccctaatctccaatcacttgctgaatcagcactgccctcctccctctctcacctgaccatctcctattgccctaatctccaatcactttcagaatcagcactgccctcctccctctctcacctgaccatctacaattgccctaatctccaatcactttcagaatcagcactgccctcctccctctctaaattatctatttccaaatgtccattgctcacaccactactagaatttgacaaggaggaatactggccacaaattgctcatatccccaccataaagatcaatgaggaatatatttaacaattaaaacaaatggctctccaactgatatgtggaatttgaagagcgaatacgaaaagtctggtacatgaattgtccgtaggaaatgtttctaaggtttgttgttacaggcaagtctttgagatgcgactatcaaagaagggcgattacaatcagtgtacccgctgatactatttcatgtttccagtgcaagcctcttttgtaagttgacaaactcgattagttaatatgtttcggactcaactagtggttagagtactcattttgtaagacttgtgtacagaaaatcaaaatagaattataactcgtg

SEQ ID NO: 5 - DNA, 21 bp, Artificial Sequence
  M18F marker
caatgataaatcgtggttggt

SEQ ID NO: 6 - DNA, 25 bp, Artificial Sequence
  M18R marker
gtcatcatatttaccaatcaatcac

SEQ ID NO: 7 - DNA, 24 bp, Artificial Sequence
  M22F Marker
cttgtgatgaatgttgagatgtac

SEQ ID NO: 8 - DNA, 25 bp, Artificial Sequence
  M22R Marker
gtcaactatcaatactgcttttatc

SEQ ID NO: 9 - DNA, 24 bp, Artificial Sequence
  M27F Marker
aaagccttttaactctcttaaaag

SEQ ID NO: 10 - DNA, 20 bp, Artificial Sequence
  M27R Marker
ctcagggcaatttctaatgg

SEQ ID NO: 11 - protein, 1246 aa, Solanum lycopersicum
  Sw-5
MAENEIEEMLEHLRRIKSGGDLDWLDILRIEELEMVLRVFRTFTKYNDVLLPDSLVELTKRAKLIGEILHRLFGRIPHKCKTNLNLERLESHLLEFFQGNTASLSHNYELNNFDLSKYMDCLENFLNDVLMMFLQKDRFFHSREQLAKHRSIKELKIVQKKIRFLKYIYATEINGYVDYEKQECLENRIQFMTNTVGQYCLAVLDYVTEGKLNEENDNFSKPPYLLSLIVLVELEMKKIFHGEVKASKFTQSKTFKDKKLPKGFSHHLHNLLMYLRNKKLENFPNNIAAQNIDVAIEFLLVFLDADVSNHVINGNWLKKVLLKVGAIAGDILYVIQKLLPRSINKDETSNISLCSIQILEKTKDLKAQVETYYKSLKFTPSQFPTFGGLSFLNSLLRKLNEMSTSKSGLGFLMKPLLGNLEKELSSLTSILEKELSSIFRDVVHHEHNIPKDLQRRTINLSYEAEVAIDSILAQYNAFLHIFCSLPTIVKEIKQINAEVTEMWSADIPLNPHYVAAPLKHLPDRHSNLVTDEEVVGFENKAEELIDYLIRGTNELDVVPIVGMGGQGKTTIARKLYNNDIIVSRFDVRAWCIISQTYNRRELLQDIFSQVTGSDDNGATVDVLADMLRRKLMGKRYLIVLDDMWDCMVWDDLRLSFPDDGIRSRIVVTTRLEEVGKQVKYHTDPYSLPFLTTEESCQLLQKKVFQKEDCPPELQDVSQAVAEKCKGLPLVVVLVAGIIKKRKMEESWWNEVKDALFDYLDSEFEEYSLATMQLSFDNLPHCLKPCLLYMGMFSEDARIPASTLISLWIAEGFVENTESGRLMEEEAEGYLMDLISSNLVMLSKRTYKGRVKYCQVHDVVHHFCLEKSREAKFMLAVKGQYIHFQPSDWKGTRVSFSFSEELSKFASLVSKTQKPFHQHLRSLITTNRAKSINDIFSCQISELRLLKVLDLSSYIVEFLSLATFKPLNQLKYLAVQAFEFYFDPGSHLPHIETFIVMNLPYYDILLPVSFWEMKKLRHAHFGKAEFDKQGLSEGSSKLENLRILKNIVGFDRVDVLSRRCPNLQQLQITYFGNNEEPFCPKLENLTQLQQLQLSFARPRTLSGLQLPSNLNKLVLEGIHIGCVIPFIAGLPSLEYLQLHDVCFPQSEEWCLGDITFHKLKLLKLVKLNISRWDVSEESFPLLETLVIKKCIDLEEIPLSFADIPTLEQIKLIGSWKVSLEDSAVRMKEEIKDTEGCDRLHLVKQRSD

SEQ ID NO: 12 - DNA, 3741 bp, Solanum lycopersicum
  Sw-5 cDNA
atggctgaaaatgaaattgaggaaatgttagagcacctgagaaggatcaagagtggaggtgatctggattggctcgacatattgcgaattgaggaacttgaaatggtgctaagagtttttagaacctttacaaagtataatgatgttcttttgcctgattccttagtcgaactcacaaagagggccaaattgattggggaaatacttcaccggttgtttggtaggattccacataaatgtaaaactaaccttaatctagaaaggctagaatcacatttgttggaattctttcaaggtaatacggcaagtttaagtcacaattatgagttgaataattttgatctgtcgaaatatatggattgtctggaaaattttctaaatgatgtactgatgatgttcttgcaaaaggataggttcttccattccagagaacaacttgcaaaacatcgatcaataaaggaactgaaaattgttcaaaagaaaataagatttttgaaatacatatatgccacagagataaatggttatgtcgactatgagaagcaggaatgtttggagaatcgaattcagttcatgactaacactgtgggacaatattgtttggcagtattagattatgtcactgagggtaaacttaatgaagaaaatgacaactttagtaaacctccttacctattatcattgattgtgttagtggagctggaaatgaagaagatttttcatggtgaagtaaaggcttcaaagtttactcaatcaaaaactttcaaggacaagaaattaccaaaaggattttcacatcatctccacaatctgttgatgtatctcagaaacaaaaagctcgagaattttcctaataatatcgctgctcaaaatattgatgtggcaatagagttcttgttggttttccttgatgctgatgtgtcaaatcatgttattaatggtaactggttgaaaaaggtcttgttaaaggttggagctatagcgggtgatattctatatgtaattcaaaagcttcttcctagatcaataaacaaagatgaaactagcaacataagtctttgctcaatacagatattggagaagactaaagatctgaaggcacaagttgagacgtactacaaatccttaaaatttactccatctcagttccccacctttggtggattgagctttctgaattctcttttaaggaaactgaatgagatgtcgacatctaagtccggattaggtttcctgatgaaacctcttttagggaatttggagaaagagctatcatctcttacatccattttagagaaggagctctcatccattttccgtgatgtcgtgcaccatgaacataacattcctaaagatcttcagaggcgtaccatcaatttgtcatatgaggctgaggttgctattgattctattcttgctcagtataatgcttttttgcatattttttgctcacttcctacaattgtaaaagagatcaagcaaattaatgcagaggtgactgagatgtggtcagcggacattcctcttaatcctcactatgtggctgctccattaaaacatctgccggatcgacatagcaatcttgtaactgatgaggaggtagtgggttttgagaataaagcagaagaactaattgattatctgattagaggtacaaatgagctagacgttgtcccaattgtaggcatgggaggacaagggaaaacgacaattgctagaaagttgtacaataatgacattattgtttctcgctttgatgttcgagcatggtgcatcatttctcaaacgtataatcggagagagttattacaagatattttcagtcaagttacaggttccgacgacaatggagctacggttgatgttcttgccgacatgttgaggagaaaattaatgggaaagagatatctcattgtattggatgatatgtgggattgtatggtatgggatgacttaaggctttcttttccagatgatggaattagaagcagaatagtcgtaacaactcgacttgaagaagtgggtaagcaagtcaagtaccatactgatccttattctcttccattcctcacaacagaagagagttgccaattgttgcagaaaaaagtgtttcaaaaggaagattgcccgcctgaactacaagatgtgagtcaagcagtagcagaaaaatgcaaaggactgcccctagtggttgtcttggtagctggaataatcaaaaaaaggaaaatggaagaatcttggtggaatgaggtgaaagatgctttatttgactatcttgacagtgagttcgaagaatacagtctggcaactatgcagttgagttttgataacttaccccactgtttaaagccttgtcttctttatatgggaatgttttcggaggacgcaagaattccagcatctacattgataagtttatggattgctgaaggattcgtggagaacactgaatctgggagattaatggaagaggaagctgaaggttacttgatggatctcattagcagtaacttggtaatgctttcaaagagaacttataagggtagagtcaaatactgtcaggttcatgatgttgtgcatcacttttgcttggaaaagagtagagaagcaaagtttatgcttgcagtgaagggtcaatatatccattttcaaccttcggattggaagggaactcgagtgagcttcagttttagtgaagagctttccaagtttgcatctctggtctccaaaacacagaagcctttccatcaacacttgaggtcattgataacgaccaatcgagcaaaatctattaatgatattttctcctgtcagattagtgaattgcgacttcttaaagtcttggatttgagttcttatattgtggagtttttgtcgttagctacattcaaaccactaaatcagctgaagtacctcgcagttcaggcttttgaattctattttgatccaggatcacatcttccccatatagaaactttcattgtaatgaatcttccttattatgatatattgttaccagtgtctttttgggaaatgaaaaaattaaggcatgctcattttggtaaggctgaatttgacaagcaggggctctctgaaggatcctctaaattggaaaatttgaggatattaaagaatattgttggatttgatagggtggatgtgttatcaaggaggtgtcctaatcttcaacaacttcaaatcacatattttgggaataatgaagagcctttttgtcccaaattggagaatcttacccagcttcaacaacttcaactttcctttgcgcgtccccgcactctatccgggttacagttgccttcaaatttaaataagttggtacttgaaggaattcatataggatgtgttattcccttcattgcgggactaccaagcctggaatatctccagttacatgatgtgtgttttcctcaatcagaagagtggtgccttggagatatcacgttccataaacttaagttgttgaaactggtaaagttaaatatatcaaggtgggatgtctcagaggaatcatttccgttgcttgaaacactcgttataaagaagtgcattgacctagaggagattccacttagctttgctgatattccaacattggaacagattaaattgattgggtcctggaaagtatctctggaggattcagctgtgagaatgaaggaagaaatcaaagacactgaaggatgtgatcgtttacacctcgtcaaacaacgctcagattga

SEQ ID NO: 13 - DNA, 300 bp, Artificial Sequence
  VIGS-1 construct
gcccacataaaaacattaaagaagtcaaaatcagtggatatagagggacaaacttccccaattgggtagctgatcctctgtttcttaagctagtgaaattgtctcttaaaaactgcaagaactgttattccttgccagcactaggacaactcccttgtttgaaattcctttctattagagggatgtatggaataagagtggtgacggaagaattctatgggagattgtcctccaaaaagccctttaactgtctagagaagcttgaatttgaagatatgacggagtggaagcaatggcacg

SEQ ID NO: 14 - DNA, 302 bp, Artificial Sequence
  VIGS-2 construct
ggggatgtatggcataatagaggtgaaggaagaattctatggtagtttgtcctccgaaaagccttttaactctcttgtggagcttagatttaaagatatgcctgagtggaagcaatggcacacactaggaattggagagttccctacacttgagaaccttttaatagaaaattgccctgatctcagtttggagacacccatccaattttcaagtttaaaaaggtttagagtcgttggttgtccagttgtttttggtgatgctcagctgtttagatcccaacttgaagcaatgaagcagattg

SEQ ID NO: 15 - DNA, 301 bp, Artificial Sequence
  VIGS-3 construct
ctatggtagtttgtcctctgaaaagccttttaactctcttaaaaggcttgaatttaaagatatgacggagtggaagcaatggcacactctaggaattggagagttccctacacttgagaacctttccattagaaattgccctgagctcagtttggagagacccatacaattttcaagtttaaaagtgtttcaagtagttggttgtccagttgtttttgatgatgctcaactgtttagatcccaacttgaggcaatgaagcagattgaggaaatatatatcagtggttgtaactctgttacc